(19) United States
(12) Patent Application Publication
(10) Pub. No.: US 2005/0273057 A1
(43) Pub. Date: Dec. 8, 2005

(54) Catheter insertion apparatus with a needle tip protective system

(76) Inventor: Sergey Popov, Beer-Sheva (IL)

Correspondence Address: RENNER, OTTO, BOISSELLE & SKLAR, LLP, Nineteenth Floor, 1621 Euclid Avenue, Cleveland, OH, 44115-2191, US

(21) Appl. No.: 11/115,037

(22) Filed: Apr. 26, 2005

Related U.S. Application Data
(60) Provisional application No. 60/565,505, filed on Apr. 26, 2004.

(51) Int. Cl. A61M 5/178

(52) U.S. Cl. 604/164.08

(57) ABSTRACT

The present invention relates to a catheter insertion apparatus with a needle protective system to protect a user from an inadvertent needle stick. One aspect of the invention provides for withdrawing a contaminated needle into a protected mode within the handle of the catheter apparatus. Another aspect of the invention provides for a frangible coupling link that prevents fixation of the apparatus into the duty ready mode after the needle has entered the protected mode. Another aspect of the invention relates to providing a transport mode whereby the apparatus is in a compact state with the catheter unit and the needle unit positioned inside the hollow handle, thereby reducing the costs associated with packaging and transportation.

### RELATED APPLICATION DATA

This application claims the benefit of U.S. Provisional Application No. 60/565,505, filed Apr. 26, 2004, which is incorporated herein by reference.

### BACKGROUND OF THE INVENTION

1. Field of Invention

The present invention relates generally to catheter placement devices for intravascular catheterization and, more particularly to, a catheter insertion apparatus for peripheral blood vessel catheterization with a needle tip protective system and a shortened length in the transport mode.

2. Description of the Related Art

Medical personnel routinely use syringes for a variety of purposes, including administering medication, for intravenous blood collection, and for inserting catheter devices. There are a number of risks associated with using syringes to perform these tasks. One risk is that of the medical personnel sticking themselves with a needle contaminated from intravenous fluids from a patient. This risk is potentially grave for medical personnel attending to patients having contagious and life threatening diseases such as Acquired Immune Deficiency Syndrome (AIDS) and Hepatitis.

Various retractable devices have been developed to protect medical personnel from inadvertent needle sticks. U.S. Pat. No. 4,507,117 to Vining et al. describes a needle/syringe device wherein the needle is fixedly projected from the syringe by engaging a pair of locking members formed on the syringe plunger with a pair of recessed portions formed on the needle mounting base, and then lockingly engaging a second pair of locking members formed on the mounting base with a second pair of recessed portions formed on the neck of the syringe barrel. The plunger must be rotated clockwise to lock each pair of locking members to their respective recessed portions. To fill the syringe with medication, the plunger is unlocked from the mounting base and drawn back in the same manner as a conventional syringe. The needle is retracted after injection by unlocking the base from the neck and drawing back on the plunger.

U.S. Pat. No. 4,988,339 to Vadher describes various retractable needle/syringe devices for blood collection, catheterization and medicinal injection procedures. The devices include a needle unit in the form of a syringe and a catheter unit received in an oblong hollow handle with a retracting spring disposed in the distal end of the handle, and a pusher of the needle unit, disposed axially at the proximal end of the handle face. The pusher is designed for manual transposition of the needle unit from the transport mode into the duty ready mode. The hollow handle is provided with slots for the transposition inside the manual controlled drive of a syringe piston and trigger member. The latter is designed for control of the needle unit retraction into the duty ready mode.

One disadvantage of the device of the '339 patent is its great length caused by the needle unit pusher, which, in the transport mode, protrudes in the proximal direction and thereby increases the length of the device. Another disadvantage of the device is the possibility of the needle unit exiting from the protection mode as a result of the action onto accessible pusher and fixation means, which allows reusing the device. Another disadvantage is the absence of direct access to the catheter hub in the duty ready mode (as shown in FIG. 5 of the '339 patent). The absence of direct access to the catheter hub impedes the control of the catheter by the user during catheter insertion into the patient's vein. Another disadvantage is the mutual disposition of the handle, the trigger member, and the pusher effectively preventing a user to control the device by the same hand that holds the device. Another disadvantage of this device is that it includes many details (e.g., the proximal lid of the handle and the pin of the syringe piston drive), which require separate fabrication and assembly. Likewise, the device includes other parts and details, such as the many slots and notches, which significantly complicate their fabrication (e.g., handle, syringe barrel, and piston). As a result, the manufacturing cost of the device grows substantially.

U.S. Pat. No. 6,086,563 to Moulton et al. discloses an apparatus having multiple modes (e.g., the transport, duty ready and retraction modes). The apparatus has increased length in the transport mode because the needle cover protrudes distally beyond the hollow handle and the trigger member is disposed at the distal end of the hollow handle beyond the needle. The apparatus also has a large number of details including the needle cover, a proximal lid of the hollow handle, a rubber cuff of the needle hub, a complex needle hub with the cantilever arm, a recess for the needle cover engagement, a complicated flash chamber, and the holder of the rubber cuff. The increased length, fabrication complexity and considerable material consumption lead to an increase of manufacturing cost associated with the apparatus.

U.S. Pat. No. 4,747,831 to Kulli discloses a needle/handle assembly wherein the needle lockingly projects outwardly from a bore formed in the handle. When the catheter has been placed, a spring activated retraction mechanism provided for the handle can be operated to automatically retract the needle into the handle. A disadvantage of this device is that the needle is constantly in a projected state outside of the handle prior to use. As a result, there exists a greater risk of potential contamination to the needle from dropping or otherwise mishandling the device, and thus, a greater risk to persons operating the device of coming into contaminating contact with the needle. Another disadvantage is increased length in the transport mode, thereby requiring increased transportation expenses. Another disadvantage is the inability to control the device with one hand. Still another disadvantage is the high probability of inadvertent retraction of the needle into the protective state.

There is a strong need in the art for an apparatus that overcomes the above described limitations in the art.

### SUMMARY OF THE INVENTION

One aspect of the invention relates to a catheter insertion apparatus, including: a housing, a needle unit operable to guide a catheter into an associated patient, a retraction mechanism to withdraw the needle unit to a protected location within at least a portion of the housing, and a selectively operable coupling to the needle unit in position for use and including a one use only unlocking device.

Another aspect of the invention relates to a catheter insertion apparatus, including: a catheter unit including a catheter tube and a catheter hub; a needle unit including a needle and a needle hub, wherein the needle hub is secured to a proximal end of the needle and the needle unit is operable to guide the catheter tube into an associated patient; a handle having a distal end and a proximal, wherein the handle serve as a guide and a receptacle for the needle unit and the catheter unit in a transport mode, a duty ready mode, and a protection mode; wherein in the transport mode the needle unit and the catheter unit are disposed within the handle, wherein in the duty ready mode the needle unit is releasably affixed to the handle adjacent to the handle distal end and the catheter hub at least partly protrudes out of said handle in a distal direction thereby providing an associated user with immediate access and control of the catheter hub by an associated hand of the associated user, and wherein in the protection mode the needle unit is disposed within the handle and is protected from accidental contact with associated persons; a retraction mechanism to retract the needle unit from the duty ready mode to the protection mode; and a selectively operable coupling to the needle unit in position for use and including a one use only unlocking device.

Another aspect of the invention relates to a catheter insertion apparatus including: a handle including a longitudinal internal cavity with a longitudinal slot in a wall of the handle, wherein the handle guides and houses a needle unit and a catheter unit; a hub engagement member and a handle engagement member for coupling the needle unit to the handle adjacent to a handle distal end of the handle in a duty ready mode, wherein the hub engagement member includes a control portion, which is disposed on an exterior portion of handle and in the duty ready mode is accessible for control by an associated hand of an associated user holding the handle, whereby the control portion is connected by a frangible coupling to be rendered inoperable by an associated force imparted by the associated user applied to the control portion for retracting the needle unit into a protection mode; and the handle engagement member includes at least one resilient latch element located on a lateral wall of the longitudinal slot, the at least one resilient latch element is pliant in a transversal direction and allows unhampered transposing of the needle unit and catheter unit from a transport mode into a duty ready mode, wherein the handle engagement member engages with the hub engagement member, thereby preventing retraction of the needle unit into the protection mode and the duty ready mode; and the handle engagement member supports at least a portion of the frangible coupling link during its breaking off, wherein a site of the breaking off is located lower than the handle engagement member and the breaking off leads to disengagement of the engagement member resulting in unhampered retraction of the needle unit into the protection mode.

Another aspect of the invention relates to a method for withdrawing a needle, the method including activating a retraction mechanism by applying a force, while holding a catheter insertion apparatus in one hand, breaking a frangible coupling, and retracting at least a portion of a needle into a handle.

Another aspect of the invention relates to a catheter insertion apparatus apparatus, including: a needle unit including a needle with a needle distal sharp point and a needle proximal end, a needle hub attached to the needle proximal end, and a flash chamber located within the needle hub; a catheter unit including a catheter tube and a catheter hub affixed to a catheter tube proximal end, wherein the needle is capable of traversing at least a portion of the catheter tube; a handle having a longitudinal internal cavity for guiding and housing the needle unit; a security zone located at the handle distal end, wherein the needle distal sharp point is protected from contact with an associated person while located in the security zone; mutual dispositions of the needle unit, catheter unit, and handle including a duty ready mode and a protection mode, wherein in the duty ready mode the needle unit is releasably affixed to the handle adjacent to a handle distal end; wherein in the protection mode the needle distal sharp point is positioned in the security zone; a hub engagement member and a handle engagement member for coupling the needle unit to the handle adjacent to the handle distal end in the duty ready mode; at least one of the hub engagement member and the handle engagement member is an active engagement member having a control portion, which is disposed outside of the handle and in the duty ready mode accessible for control by the same user hand which holds apparatus, and the active engagement member is connected by a frangible coupling link to be rendered inoperable by an associated force imparted by the associated user applied to the control portion for retracting the needle unit into the protection mode; and a resilient retracting member having a maximum stressed state in the duty ready mode and positioned beyond a blood flow for retraction of the needle unit from the duty ready mode into the protection mode.

Another aspect of the invention relates to a catheter insertion apparatus apparatus, includes: a needle unit including a needle with a needle distal sharp point and a needle proximal end, a needle hub attached to the needle proximal end, and a flash chamber located within the needle hub; a catheter unit including a catheter tube and a catheter hub affixed to a catheter tube proximal end, wherein the needle is capable of traversing at least a portion of the catheter tube; a handle having a longitudinal internal cavity for guiding and housing the needle unit; a security zone located at the handle distal end, wherein the needle distal sharp point is protected from contact with an associated person while located in the security zone; mutual dispositions of the needle unit, catheter unit, and handle including a duty ready mode and a protection mode, wherein in the duty ready mode, the needle unit is releasably affixed to the handle adjacent to a handle distal end; wherein in the protection mode the needle distal sharp point is positioned in the security zone; a hub engagement member and a handle engagement member for coupling the needle unit to the handle adjacent to the handle distal end in the duty ready mode; at least one of the hub engagement member and the handle engagement member is an active engagement member having a control portion located outside of the handle and in the duty ready mode accessible for control by the same user hand which holds apparatus, wherein control of the active engagement member is actuated by a force applied from the associated user to the control portion which retracts the needle unit into the protection mode, wherein the applied force is distinct from the movement direction of the catheter unit relative to the needle; and a resilient retracting member having a maximum stressed state in the duty ready mode and positioned beyond a blood flow for retraction of the needle unit from the duty ready mode into the protection mode.

Another aspect of the invention relates to a catheter insertion apparatus apparatus, includes: a needle unit including a needle with a needle distal sharp point and a needle proximal end, a needle hub attached to the needle proximal end, and a flash chamber located within the needle hub; a catheter unit including a catheter tube and a catheter hub affixed to a catheter tube proximal end, wherein the needle is capable of traversing at least a portion of the catheter tube; a handle including a longitudinal internal cavity with a longitudinal slot in a wall of the handle, wherein the handle guides and houses the needle unit and the catheter unit; a security zone located at a handle distal end, wherein the needle distal sharp point is protected from contact with an associated person while located in the security zone; mutual dispositions of the needle unit, catheter unit, and handle including a transport mode, a duty ready mode and a protection mode, wherein in the transport mode, the needle unit and the catheter unit are disposed in the longitudinal internal cavity of the handle, the needle is disposed inside the catheter tube so that the needle distal sharp point protrudes distally from the distal end of the catheter tube, the catheter unit is disposed in a proximal position, the needle hub is disposed near a handle proximal end, and the needle distal sharp point is disposed in the security zone; wherein in the duty ready mode the needle unit is releasably affixed to the handle adjacent to a handle distal end; wherein in the protection mode the needle distal sharp point is positioned in the security zone; a duty fixation means for releasably affixing the needle unit to the handle adjacent to the handle distal end in the duty ready mode, including a hub engagement member disposed on the needle hub and a handle engagement member disposed on the handle, wherein the hub engagement member is connected to the needle hub by a frangible coupling link and has a control portion extending through the longitudinal slot of the handle, wherein the control portion is adapted for the control by an associated user's finger for manual transposition of the catheter unit and needle unit from the transport mode into the duty ready mode, and in the duty ready mode the control portion is accessible for control by the same user hand, which holds the catheter insertion apparatus, to break off the frangible coupling link by a force applied by the associated user to the control portion for disengaging the engagement members and retracting the needle unit into the protection mode; and a resilient retracting member having a maximum stressed state in the duty ready mode and positioned beyond a blood flow for retraction of the needle unit from the duty ready mode into the protection mode.

Other apparatuses, methods, features, and advantages of the present invention will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Likewise, elements and features depicted in one drawing may be combined with elements and features depicted in additional drawings. Moreover, in the drawings, like reference numerals designate corresponding parts throughout the several views.

FIG. 1 illustrates a longitudinal view, partly in section, of the catheter insertion apparatus in the transport mode;

FIG. 2 illustrates a general side view of the catheter insertion apparatus in the transport mode;

FIGS. 3, 4 and 5 illustrate respective transverse section views of the catheter insertion apparatus in the transport mode looking in the direction of the arrows 3-3, 4-4 and 5-5, respectively;

FIG. 6 illustrates a general top view of the catheter insertion apparatus in the transport mode;

FIG. 7 illustrates an enlarged partial top view of the catheter insertion apparatus in the zone of the duty fixation element location;

FIG. 8 illustrates a side view, partly in section, of the catheter insertion apparatus in the duty ready mode;

FIG. 9 illustrates a transverse view, partly in section, of the catheter insertion apparatus in the protection mode;

FIG. 10 illustrates a longitudinal view, partly in section, of the catheter insertion apparatus in the transport mode with a spring retraction mechanism;

FIG. 11 illustrates a side view, partly in section, of the catheter insertion apparatus of FIG. 10 in the duty ready mode;

FIG. 12 illustrates a transverse view, partly in section, of the catheter insertion apparatus of FIG. 10 in the protection mode;

FIG. 13 illustrates a longitudinal view, partly in section, of the catheter insertion apparatus in the transport mode;

FIGS. 14 and 15 illustrate a side view of the catheter insertion apparatus in the transport mode;

FIGS. 16 and 17 illustrate a transverse view, partly in section of the catheter insertion apparatus of FIG. 13 in the transport mode;

FIG. 18 illustrates a side view, partly in section, of the catheter insertion apparatus of FIG. 13 in the duty ready mode;

FIG. 19 illustrates a transverse section view of the catheter insertion apparatus in the transport mode looking in the direction of the arrows 19-19, as shown in FIG. 18; and

FIG. 20 illustrates a transverse view, partly in section, of the catheter insertion apparatus in the protection mode.

### DETAILED DESCRIPTION OF THE INVENTION

A catheter insertion apparatus 10 (also referred to as a catheter introducer) for intravascular catheterization, which is illustrated in the drawings, generally includes a catheter unit with a catheter tube and a catheter hub, a needle unit with a tubular needle and a needle hub, and a hollow handle capable of receiving at least a portion of the catheter unit and needle unit.

The catheter insertion apparatus 10 has multiple modes or states (also referred to as “positions”), which are used to indicate the relative positions and relationships among the respective parts, during normal operation. For example, in normal use, the catheter insertion apparatus 10 has three general modes (positions): the transport mode, the duty ready mode, and the protection mode. The transport mode is the mode in which the catheter insertion apparatus 10 ordinarily may be stored and/or transported from place to place before it is used. In the duty ready mode, the catheter insertion apparatus 10 is ready for use. In the protection mode, the needle unit is withdrawn after use and an individual is generally protected from exposure and/or inadvertent sticking from the needle. These labels (transport mode, duty ready mode and protection mode) are used herein for convenience of summary and not intended to be limits on the description of the invention, which is described in greater detail below.

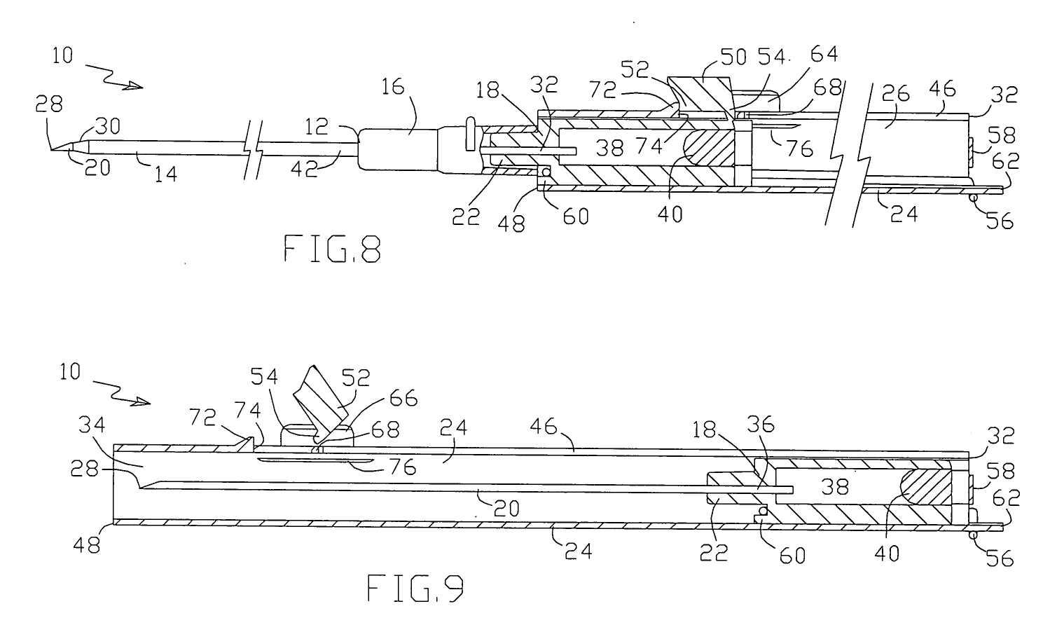

FIGS. 1-7 illustrate the catheter insertion apparatus 10 in the transport mode. FIGS. 8 and 9, respectively illustrate the catheter insertion apparatus 10 in the duty ready mode and in the protective mode (e.g., after use). A retraction mechanism of the catheter insertion apparatus may be operated manually to cause a withdrawing of the needle unit at the end of or after use of the catheter insertion apparatus. The control mechanism, which is described below, may allow for manual operation using the same hand that holds the catheter insertion apparatus during insertion of the needle unit into a patient, may be restricted to a single use to restrict the catheter insertion apparatus a one time use; and in cooperation with a needle unit withdrawing force mechanism operates to assure that in the process of withdrawing the needle unit, the applied force is in a direction distinct from the direction of inserting the needle into the patient.

Referring to FIG. 1, a catheter insertion apparatus 10 generally includes a catheter unit 12 with a catheter tube 14 and a catheter hub 16, a needle unit 18 with a tubular needle 20 and a needle hub 22, as well as a hollow handle 24. As shown in FIG. 1, in the transport mode, catheter unit 12 and needle unit 18 are disposed in internal cavity 26 within the axial limits of handle 24. The needle 20 is disposed inside catheter tube 14 so that needle distal sharp point 28 protrudes distally from the distal end 30 of the catheter tube 14. The catheter unit 12 is disposed in the extreme proximal position of the handle 24, the needle hub 22 is disposed near handle proximal end 32, and needle distal sharp point 28 is disposed in security zone 34.

As shown in FIG. 1, in the transport mode, at least a portion of the needle unit 18 and the catheter unit 12 are housed within the handle 24. Thus, in the transport mode, the catheter insertion apparatus 10 has a reduced length, which provides advantages in the form of lower packaging and transportation costs. In the duty ready mode, discussed below, the catheter insertion apparatus 10 has a relatively long length, approximately equal to the sum of the length of the hollow handle 24 and the length of the catheter unit 12. In the protection mode, also discussed below, the apparatus is shorter than the duty ready mode because the needle unit 18 is housed inside the hollow handle 24.

Referring to FIG. 1, the needle unit 18 generally includes a needle 20 having a needle distal sharp point 28 and a needle proximal end 36, the needle hub 22 attached to the needle proximal end 36. The needle hub 22 includes a flash chamber 38 located within needle hub 22 and a porous membrane 40 permeable for air and non-permeable for blood. The catheter unit 12 generally includes a catheter tube 14 and a catheter hub 16 affixed to the catheter tube proximal end 42. The handle 32 of the catheter insertion apparatus 10 includes a longitudinal internal cavity 26, which serves as a guide and a receptacle for needle unit 18 and catheter unit 12. A longitudinal slot 46 is located in the wall of internal cavity 26. A security zone 34 is positioned at handle distal end 48 for protecting medical personnel and patients from inadvertent contact with the needle distal sharp point 28.

The catheter insertion apparatus 10 further includes a control mechanism 50 and a hub engagement member 52. The control mechanism 50 provides for control and operation for the catheter insertion apparatus 10 to be manipulated by the same hand of a user, such as a nurse, doctor, medical technician, or the like that may be holding the apparatus 10.

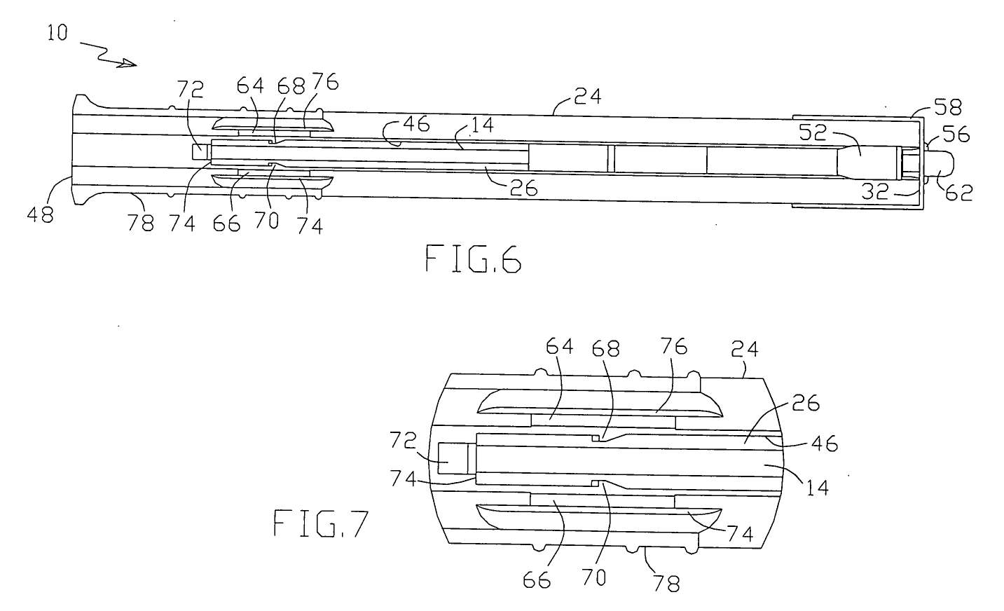

As discussed more fully below, the catheter insertion apparatus 10 further includes a retraction mechanism for retraction of the needle unit 18 into the handle 24 after use. The control mechanism a frangible coupling link 54, a resilient member 56, proximal stopping element 58 and protrusions 60, 62. In addition, the catheter insertion apparatus 10 includes projections 64 and 66 (shown in FIG. 2), handle engagement members 68 and 70 (shown in FIG. 6), distal stopping element 72, longitudinal slot 46 and lateral longitudinal slits 74 (shown in FIG. 2) and 76.

Referring to FIG. 2, an external view of the catheter insertion apparatus 10 in the transport mode is illustrated. The handle 24 includes a gripping portion 80 located near the distal end 48 of the handle 32. The finned gripping portion 78 provides a convenient mechanism for handling the catheter insertion apparatus 10 due to its overall resistance to slipping and ability to fit comfortably in the user's hand.

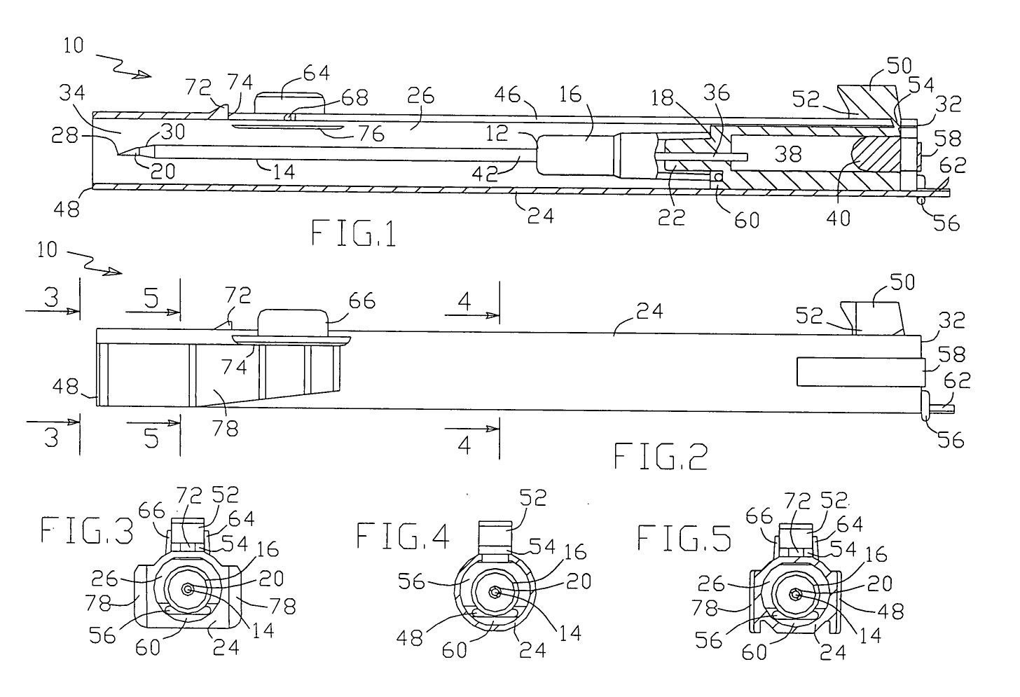

FIGS. 3-5 illustrate cross-sectional views of the catheter insertion apparatus 10 along the handle 24, as shown in FIG. 2. Referring to FIGS. 3-5, needle 20, catheter tube 14, and catheter hub 16 are shown coaxially positioned in internal cavity 26 of the handle 24. In addition, frangible coupling link 54 is illustrated along with resilient member 56 and protrusion 60. FIGS. 3 and 5 further illustrate the gripping portion 47 of the handle 24, projections 64 and 66, hub engagement member 52 and distal stopping element 72. FIG. 4 also illustrates the hub engagement member 52 associated with the control mechanism 50.

FIGS. 6 and 7 illustrate a top view of the of the catheter insertion apparatus 10. As shown, the catheter insertion apparatus 10 includes a retaining mechanism in the form of two projections 64 and 66 located on an outer surface of handle 24 on either side of longitudinal slot 46 at longitudinal slot distal end 74 and adapted for lateral forcing against hub engagement member 52 in the duty ready mode. The friction forces arising between hub engagement member 52 and projections 64 and 66 permit the functional motions of hub engagement member 52 but retain the hub engagement member 52 on handle 24 after breaking off frangible coupling link 54. This eliminates undesirable and uncontrolled falling of the hub engagement member 52 into the patient's surroundings and improves the operation safety of the catheter insertion apparatus 10, as discussed more fully below.

Handle engagement members 68 and 70 are made as resilient latch elements located on a lateral wall of longitudinal slot 46 and pliant in the transverse direction due to lateral longitudinal slits 74 and 76. Due to their pliability, handle engagement members 68 and 70 permit unhampered transposing of needle unit 18 and catheter unit 12 from the transport mode into the duty ready mode wherein handle engagement members 68 and 70 engage with hub engagement member 52, thereby preventing the retraction of needle unit 18 proximally into the protection mode, while distal stopping element 72 restricts the movement of needle unit 18 in the distal direction.

From the transport mode, the catheter insertion apparatus 10 is easily positioned into the duty ready mode prior to use. In particular, the catheter insertion apparatus 10 is adjusted so that the sharp distal point 28 of the needle 20 extends beyond the distal end of the catheter tube 30 and the catheter tube 14 extends beyond the distal end 48 of the hollow handle 24, as shown in FIG. 8. The control mechanism 50 is generally used to place the catheter insertion apparatus 10 in the duty ready mode. The control mechanism is accessible for control by the same user hand, which holds the catheter insertion apparatus 10.

In the duty ready mode, needle unit 18 is releasably affixed to handle 24 and adjacent to handle distal end 48 by the retaining mechanism in the form of two projections 64 and 66 (shown in FIG. 7) and hub engagement member 52. Catheter hub 16 protrudes out of handle 24 in a distal direction thereby providing the user with immediate access and immediate control of the catheter hub 16 by the same user's hand which holds the catheter insertion apparatus 10.

While the catheter insertion apparatus 10 is in the duty ready mode, the user may hold the apparatus at finned gripping portion 78 of handle 24 and set the catheter unit 12 into patient's body.

After the catheter unit 12 has been properly placed in the patient, the needle unit 18 may be retracted. For example, the user may apply a force to the hub engagement member 52 to retract the needle unit 18 into the internal cavity 26 of the handle 24. The applied force is first upward and then in the proximal direction in order to break off (or snap) the frangible coupling link 54. For example, the applied force may be in a lifting, tilting, etc. type of action. The applied force is in a direction that is distinct from the movement direction of catheter unit 12 relative to needle 20 and from a direction towards the handle longitudinal axis (away from the patient). The application of the force in this manner eliminates the accidental premature activation of the retracting mechanism during the catheter advance into patient's body using needle 20 as a catheter guide. During the breaking off or snapping of the frangible coupling link 54, handle engagement members 68 and 70 serve as a support for the frangible coupling link 54. The location of the breaking (or snapping) off is located below the positions of the lower handle engagement members 68 and 70, thereby eliminating the needle unit 18 from jamming and enhancing the reliability of the needle unit retraction.

As illustrated, needle hub 18 and hub engagement member 52 are made as a single detail of fragile plastic. This aspect of the invention considerably simplifies the design and assembling of the catheter insertion apparatus 10 and reduces the detail number and ultimately the cost of the device. After breaking off hub engagement member 52, engagement members 52, 68, and 70 are disengaged and needle unit 18 is retracted into the protection mode. As stated previously, the location of the breaking off is located lower on the handle engagement members 68 and 70, thereby eliminating the needle unit 18 jamming and enhancing the reliability of retraction of the needle unit 18. The breaking off of the hub engagement member 52 essentially eliminates possible re-use of the device, as there is no possibility for the repeated transposition of needle unit 18 into the duty ready mode.

To prevent the user from being pricked or stuck by the needle sharp distal point 28, the withdrawn needle 20 is transposed into the protection mode avoiding the transmission of infection by the blood contaminated needle 20. In this withdrawn or protected mode, the needle unit 18 is disposed inside the hollow handle 24 and the needle sharp distal point 28 is disposed in a security zone 34 to avoid contact of the user with the sharp distal point 28.

Referring to FIG. 9, the catheter insertion apparatus 10 is illustrated in the protection mode. The needle distal sharp point 28 is positioned in security zone 34. The duty fixation mechanism includes hub engagement member 52 disposed on needle hub 22 and handle engagement members 68, 70 (shown in FIGS. 6 and 7) and 68 disposed on handle 24. Hub engagement member 52 is an active engagement member, which is connected with needle hub 22 by a frangible coupling link 54 and has a control mechanism 50 (shown in FIGS. 1, 2, and 8) extending to the outside through longitudinal slot 46 and adapted for the operation by user's finger for the manual transposition of catheter unit 12 and needle unit 18 from the transport mode into the duty ready mode.

The resilient retracting mechanism for the retraction of needle unit 18 from the duty ready mode into the protection mode includes resilient member 56 installed beyond a blood flow and having maximum stressed state in the duty ready mode. Resilient member 56 is made of elastic polymer like rubber in the form of a ring and secured to needle hub 18 and handle 24 by the coupling mechanism made as protrusion 62 located near handle proximal end 32 and protrusion 60 located on needle hub 18. Resilient member 56 also serves as a transport and duty ready fixation mechanism, which prevents needle unit 18 from spontaneous displacement in the distal direction from the transport and duty ready modes. Proximal stopping element 58 is preferably a sticky tape that restricts the proximal displacement of needle unit 18 beyond the scope of handle proximal end 32.

Further embodiments of the catheter insertion apparatus 110 are shown in FIGS. 10-12. Catheter insertion apparatus 110 has the resilient retracting mechanism with resilient member 148 made as a compression spiral spring disposed lengthwise in handle 124. In the transport mode, illustrated in FIG. 10, catheter hub 116 is disposed inside spring 148, and this spring allows free transposition of catheter hub 116 from the transport mode to the duty ready mode.

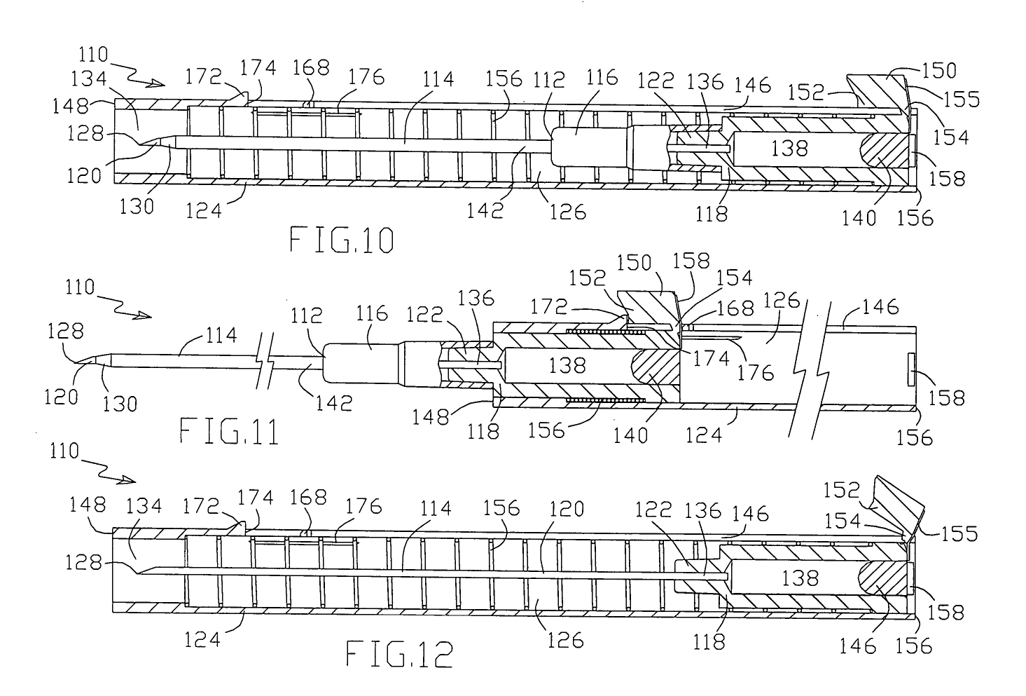

As shown in FIG. 11, in the duty ready mode, the resilient member 148 is in a maximum compressed state (as compared to the transport and protection modes). The functions and operation of resilient member 148 are identical to the resilient member 48 discussed above, except that resilient member 48 prefers a reduced length in its relaxed condition and resilient member 148 prefers an expanded length in its relaxed position. The retaining mechanism 155 of the catheter insertion apparatus 110 is made of flexible tape, which connects hub engagement member 152 with needle unit 118. As shown in FIG. 12, after breaking off hub engagement member 152, the retaining mechanism 155 retains hub engagement member 152 in the scope of catheter insertion apparatus 110, which eliminates undesirable and uncontrolled falling of the hub engagement member 152 into the patient's surroundings. Proximal stopping element 158 is made as a small projection at distal end of internal cavity 126. The walls of internal cavity 126 are somewhat pliant due to longitudinal slot 146, and proximal stopping element 158 has a sloping surface on its proximal side. These features allow needle unit 118 to overcome proximal stopping element 158 during assembly of the catheter insertion apparatus 110. Otherwise, the design and operation of catheter insertion apparatus 110 are identical to one of above catheter insertion apparatus 10 and designations of identical parts of these devices have the same two last numerals, where appropriate.

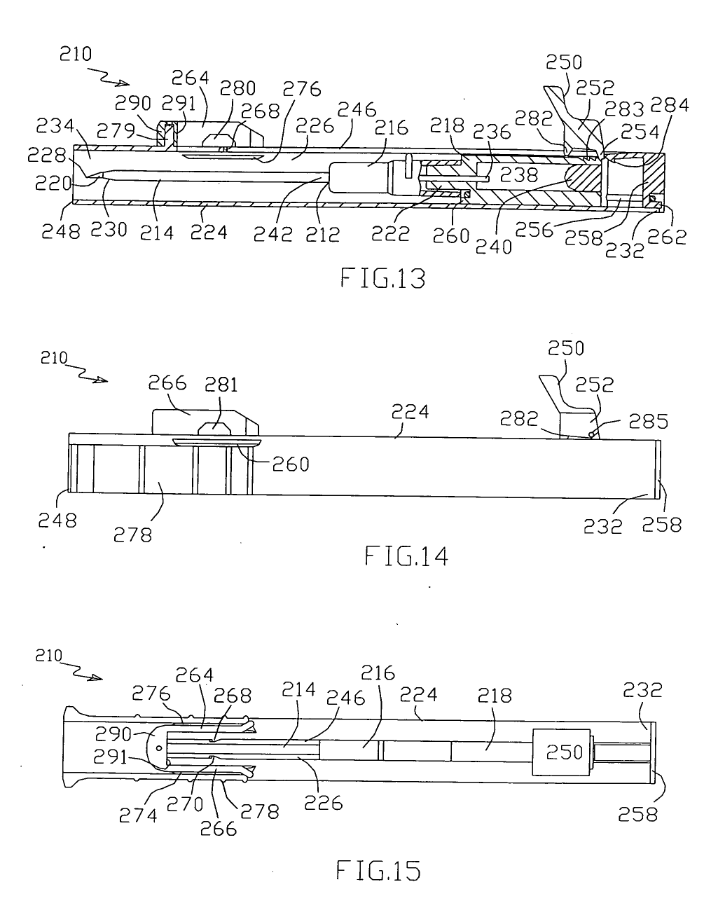

FIGS. 13-20 illustrate another embodiment of the catheter insertion apparatus 210. The catheter insertion apparatus 210 includes a retaining mechanism, a protection fixation mechanism and a proximal stopping element, that differs from the catheter insertion apparatus 10 described above. Otherwise, the catheter insertion apparatuses 10 and 210 have substantially identical design and operation, with the identical elements identified in FIGS. 13-20 using the same two last reference numbers of detail designations previously identified with reference to FIGS. 1-12.

The retaining mechanism includes two resilient plates 264, 266 located on an outer surface of handle 224 on either side of handle longitudinal slot 246, at the distal end of the compact insertion apparatus 210. Resilient plates 264, 266 can be made as elements of integral unit 290 mounted on handle 224 by any means (e.g., mounted on handle upper pin 279 and held thereon by friction). The resilient plates 264, 266 are provided with lateral sockets 280, 281 adapted for receiving lateral pins 285, 286 of hub engagement member 252 in a duty ready mode, as shown in FIGS. 18 and 19, and subsequent holding the lateral pins 285, 286 to retain hub engagement member 252 after breaking off frangible coupling link 254 and retracting needle unit 212 into the protection mode, as shown in FIG. 20. The inner distal surface 291 of integral unit 290 acts as a distal stopping element restricting the distal movement of hub engagement member 252 during transposing needle unit 212 into the duty ready mode. The described retaining mechanism relaxes demands to fabrication tolerances, eliminates increased frictions and jamming in the retaining mechanism and reliably retains hub engagement member 252 after breaking off frangible coupling link 254.

The protection fixation mechanism includes locking pawl 284 disposed on resilient leg 288, which is generally a portion of handle plug 258 and locking notches 283, which may be generally located on the proximal end of needle hub 222. In the transport mode, shown in FIGS. 13-16, distal end 289 of locking pawl 284 may rest against frangible coupling link 254, thereby preventing the displacement of needle unit 212 into the protection mode and the engagement of locking pawl 284 with locking notches 283. After breaking off frangible coupling link 254, resilient member 256 transposes needle unit 212 proximally into the protection mode, which results in the engagement of locking pawl 284 and locking notches 283 and the fixation of needle unit 212 in the protection mode, as shown in FIG. 20. Thus, needle unit 212 in the protection mode is disposed somewhat proximally with respect to its position in the transport mode. Locking notches 283 may be disposed successively along the needle hub axis, which allows advancing locking pawl 284 from one notch to another during its movement into the protection mode thereby providing a braking mechanism for needle unit 212.

Handle plug 258 can be installed at handle proximal end 232. The handle plug 258 serves as a proximal stopping element restricting the proximal movement of needle unit 212 during its retracting into the protection mode. The handle plug 258 may also include element 262, as shown in FIG. 13, of the coupling mechanism designed for securing resilient member 256 at the handle proximal end.

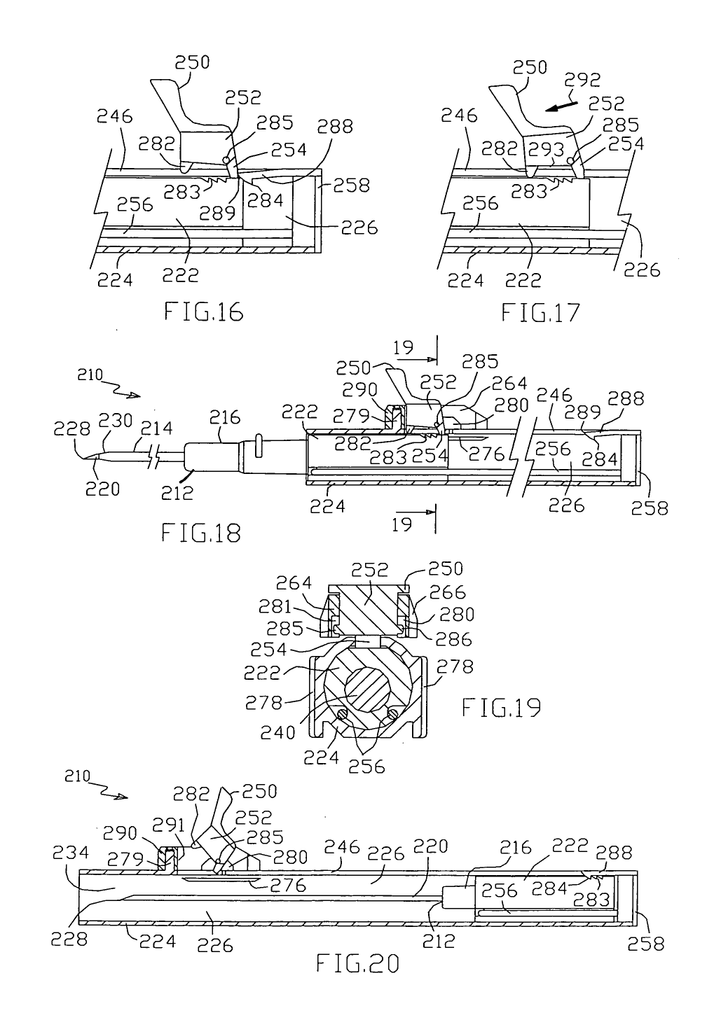

Hub engagement member 252 may also be provided with a spacer 282 in the form of projection 282 located on the lower distal surface of hub engagement member 252 and entering handle longitudinal slot 246. Spacer 282 provides a gap 293, as shown in FIG. 17, between hub engagement member 252 and handle 224 during transposing needle unit 212 from the transport mode to the duty ready mode under action of user's effort 292 to prevent friction and possible jamming between the handle 224 and the hub engagement member 252.

Although the invention has been shown and described with respect to certain preferred embodiments, it is obvious that equivalents and modifications will occur to others skilled in the art upon the reading and understanding of the specification. The present invention includes all such equivalents and modifications, and is limited only by the scope of the following claims.

1. A catheter insertion apparatus, comprising:
a catheter unit including a catheter tube and a catheter hub;
a needle unit including a needle and a needle hub, wherein the needle hub is secured to a proximal end of the needle and the needle unit is operable to guide the catheter tube into an associated patient;
a handle having a distal end and a proximal, wherein the handle serve as a guide and a receptacle for the needle unit and the catheter unit in a transport mode, a duty ready mode, and a protection mode;
wherein in the transport mode the needle unit and the catheter unit are disposed within the handle, wherein in the duty ready mode the needle unit is releasably affixed to the handle adjacent to the handle distal end and the catheter hub at least partly protrudes out of said handle in a distal direction thereby providing an associated user with immediate access and control of the catheter hub by an associated hand of the associated user, and wherein in the protection mode the needle unit is disposed within the handle and is protected from accidental contact with associated persons;
a retraction mechanism to retract the needle unit from the duty ready mode to the protection mode; and
a selectively operable coupling to the needle unit in position for use and including a one use only unlocking device.

2. The apparatus of claim 1, wherein the retraction mechanism is a resilient member.

3. The apparatus of claim 3 further including a coupling mechanism in the form of at least one protrusion located on the needle hub and another located on the handle proximal end to secure the resilient member.

4. The apparatus of claim 3, wherein the resilient member is a rubber ring secured to the needle hub and handle.

5. The apparatus of claim 1, wherein the retraction mechanism is a spring.

6. The apparatus of claim 5, wherein said spring is a compression spiral spring disposed lengthwise in the handle, wherein in the transport mode the catheter hub is disposed inside at least a portion of the spring and the spring allows free transposition of the catheter hub from the transport mode to the duty ready mode.

7. The apparatus of claim 1, wherein the one use only unlocking device is a frangible coupling.

8. The apparatus of claim 7, wherein the frangible coupling is a pivot.

9. The apparatus of claim 8, wherein the pivot is actuable away from an insertion point of the needle unit into the associated patient.

10. The apparatus of claim 9, wherein the pivot rotates away from an insertion point of the needle unit into the associated patient.

11. The apparatus of claim 1, wherein an applied force is required to activate the retraction mechanism.

12. The apparatus of claim 11, wherein the applied force is directed upward and proximally.

13. A catheter insertion apparatus comprising:
a handle including a longitudinal internal cavity with a longitudinal slot in a wall of the handle, wherein the handle guides and houses a needle unit and a catheter unit;
a hub engagement member and a handle engagement member for coupling the needle unit to the handle adjacent to a handle distal end of the handle in a duty ready mode, wherein the hub engagement member includes a control portion, which is disposed on an exterior portion of handle and in the duty ready mode is accessible for control by an associated hand of an associated user holding the handle, whereby the control portion is connected by a frangible coupling to be rendered inoperable by an associated force imparted by the associated user applied to the control portion for retracting the needle unit into a protection mode; and
the handle engagement member includes at least one resilient latch element located on a lateral wall of the longitudinal slot, the at least one resilient latch element is pliant in a transversal direction and allows unhampered transposing of the needle unit and catheter unit from a transport mode into a duty ready mode, wherein the handle engagement member engages with the hub engagement member, thereby preventing retraction of the needle unit into the protection mode and the duty ready mode; and the handle engagement member supports at least a portion of the frangible coupling link during its breaking off, wherein a site of the breaking off is located lower than the handle engagement member and the breaking off leads to disengagement of the engagement member resulting in unhampered retraction of the needle unit into the protection mode.

14. The apparatus of claim 13 further including a protection fixation mechanism for fixation of the needle unit in the protection mode, wherein the protection fixation mechanism includes a resilient locking pawl and at least one locking notch.

15. The apparatus of claim 14, wherein one locking notch is located at the handle and another locking notch located near the needle unit to provide engagement between the needle unit and the handle in the protection mode for fixation of then needle unit in the duty ready mode.

16. The apparatus of claim 13 further including a handle plug installed at a handle proximal end and serving as a proximal stopping element restricting proximal movement of the needle unit during retraction into the protection mode.

17. The apparatus of claim 16, wherein the handle plug includes at least one protrusion that secures the resilient latch element at said handle proximal end.

18. The apparatus of claim 17 further including a locking pawl on the handle plug and a locking notch located on a proximal end of the needle hub, wherein in the transport mode a distal end of the locking pawl rests against a portion of the frangible coupling link thereby preventing the displacement of the needle unit into the protection mode.

19. The apparatus of claim 18 further including the locking pawl engaging the locking notch to provide displacement of the needle unit into the protection mode and engagement of the locking pawl and notch after breaking off the frangible coupling link from the needle hub, thereby resulting in the needle unit being positioned in the protection mode disposed more proximally than when positioned in the transport mode.

20. The apparatus of claim 19, wherein a plurality of locking notches are disposed successively along the needle hub, thereby allowing positioning of the locking pawl from one locking notch to another during its movement into the protection mode.

21. The apparatus of claim 13, wherein the hub engagement member and the needle hub are made as a single detail of breakable plastic.

22. The apparatus of claim 21, wherein said hub engagement member is provided with a spacer in the form of a projection located on one of said hub engagement member and said needle hub, entering a handle longitudinal slot, wherein said hub engagement member is movable, and providing a gap between said hub engagement member and said handle during transposing said needle unit from said transport mode to said duty ready mode to prevent the friction and probable jamming between them.

23. The apparatus of claim 21, wherein there is a retaining mechanism for retaining the hub engagement member after breaking off the frangible coupling link.

24. The apparatus of claim 23, wherein the retaining mechanism includes two resilient plates located on either side of a handle longitudinal slot positioned on an outer surface of the handle, wherein said hub engagement member is movable near a distal end of the longitudinal slot, and the resilient plates are provided with lateral sockets adapted for receiving and holding lateral pins of the hub engagement member in the duty ready mode to retain the hub engagement member after breaking off the frangible coupling link.

25. The apparatus of claim 21, wherein sad spacer is located on a lower distal surface of said hub engagement member.

26. A method for withdrawing a needle, the method comprising:
activating a retraction mechanism by applying a force, while holding a catheter insertion apparatus in one hand;
breaking a frangible coupling; and
retracting at least a portion of a needle into a handle.

27. The method of claim 26, wherein the applied force is directed upward and proximally.

28. The method of claim 27, further including preventing the catheter insertion apparatus to be locked in a duty ready position after a single use.

29. The method of claim 28, further including transporting the catheter insertion apparatus in a transport mode prior to use in an associated patient.

30. A catheter insertion apparatus, comprising:
a needle unit including a needle with a needle distal sharp point and a needle proximal end, a needle hub attached to the needle proximal end, and a flash chamber located within the needle hub;
a catheter unit including a catheter tube and a catheter hub affixed to a catheter tube proximal end, wherein the needle is capable of traversing at least a portion of the catheter tube;
a handle having a longitudinal internal cavity for guiding and housing the needle unit;
a security zone located at the handle distal end, wherein the needle distal sharp point is protected from contact with an associated person while located in the security zone;
mutual dispositions of the needle unit, catheter unit, and handle including a duty ready mode and a protection mode, wherein in the duty ready mode the needle unit is releasably affixed to the handle adjacent to a handle distal end; wherein in the protection mode the needle distal sharp point is positioned in the security zone;
a hub engagement member and a handle engagement member for coupling the needle unit to the handle adjacent to the handle distal end in the duty ready mode;
at least one of the hub engagement member and the handle engagement member is an active engagement member having a control portion, which is disposed outside of the handle and in the duty ready mode accessible for control by the same user hand which holds apparatus, and the active engagement member is connected by a frangible coupling link to be rendered inoperable by an associated force imparted by the associated user applied to the control portion for retracting the needle unit into the protection mode; and
a resilient retracting member having a maximum stressed state in the duty ready mode and positioned beyond a blood flow for retraction of the needle unit from the duty ready mode into the protection mode.

31. A compact catheter insertion apparatus, comprising a needle unit that may be disposed in at least a duty ready mode and a protective mode in a handle, wherein at least one locking notch is located at the handle and another locking notch is located near the needle unit to provide engagement between the needle unit and the handle in the protection mode for fixation of the needle unit in the duty ready mode.

32. The apparatus of claim 31, wherein one locking notch is located at the handle and another locking notch located near the needle unit to provide engagement between the needle unit and the handle in the protection mode for fixation of then needle unit in the duty ready mode.

33. The apparatus of claim 32 further including a handle plug installed at a handle proximal end and serving as a proximal stopping element restricting proximal movement of the needle unit during retraction into the protection mode.

34. The apparatus of claim 31, wherein the handle plug includes at least one protrusion that secures the resilient latch element at said handle proximal end.

35. The apparatus of claim 34 further including a locking pawl on the handle plug and a locking notch located on a proximal end of the needle hub, wherein in the transport mode a distal end of the locking pawl rests against a portion of the frangible coupling link thereby preventing the displacement of the needle unit into the protection mode.

36. The apparatus of claim 35 further including the locking pawl engaging the locking notch to provide displacement of the needle unit into the protection mode and engagement of the locking pawl and notch after breaking off the frangible coupling link from the needle hub, thereby resulting in the needle unit being positioned in the protection mode disposed more proximally than when positioned in the transport mode.

37. The apparatus of claim 36, wherein a plurality of locking notches are disposed successively along the needle hub, thereby allowing positioning of the locking pawl from one locking notch to another during its movement into the protection mode.

38. The apparatus of claim 31, wherein the hub engagement member and the needle hub are made as a single detail of breakable plastic.

39. The apparatus of claim 38, wherein said hub engagement member is provided with a spacer in the form of a projection located on one of said hub engagement member and said needle hub, entering a handle longitudinal slot, wherein said hub engagement member is movable, and providing a gap between said hub engagement member and said handle during transposing said needle unit from said transport mode to said duty ready mode to prevent the friction and probable jamming between them.

40. The apparatus of claim 38, wherein there is a retaining mechanism for retaining the hub engagement member after breaking off the frangible coupling link.

41. The apparatus of claim 40, wherein the retaining mechanism includes two resilient plates located on either side of a handle longitudinal slot positioned on an outer surface of the handle, wherein said hub engagement member is movable near a distal end of the longitudinal slot, and the resilient plates are provided with lateral sockets adapted for receiving and holding lateral pins of the hub engagement member in the duty ready mode to retain the hub engagement member after breaking off the frangible coupling link.

42. The apparatus of claim 38, wherein said spacer is located on a lower distal surface of said hub engagement member.
